(19) United States
(12) Patent Application Publication
(10) Pub. No.: US 2014/0017346 A1
(43) Pub. Date: Jan. 16, 2014

(54) GELATIN-FREE, ISOMALTULOSE-CONTAINING SOFT CARAMEL

(71) Applicants: Jorg Bernard, Albsheim (DE); Kowalczyk Jorg, Eisenberg (DE)

(72) Inventors: Jorg Bernard, Albsheim (DE); Kowalczyk Jorg, Eisenberg (DE)

(73) Assignee: Sudzucker Aktiengesellschaft Mannheim/Ochsenfurt, Mannheim (DE)

(21) Appl. No.: 14/027,667

(22) Filed: Sep. 16, 2013

Related U.S. Application Data
(62) Division of application No. 10/697,800, filed on Oct. 31, 2003.

(51) Int. Cl. A23G 3/42; A61K 9/00

(52) U.S. Cl. CPC A23G 3/42; A61K 9/0056
USPC 424/742; 426/572; 426/548; 426/250; 514/289; 514/731; 514/317; 514/653; 514/535; 514/358

(57) ABSTRACT

Disclosed is a gelatin-free soft caramel including a soft caramel base mass that contains at least one polysaccharide hydrocolloid as texturing agent, a crystalline sweetener phase formed by isomaltulose, and a noncrystalline sweetener phase, wherein the caramel is gelatin-free. It can be made by providing a noncrystalline sweetener phase by dissolving at least one soluble sweetener in water, adding polysaccharide hydrocolloid, fat component, emulsifier and a part of the total amount of the isomaltulose to the noncrystalline sweetener phase, steam heating the mixture to a temperature of at least 100° C., adding the remaining isomaltulose to the heated mixture while stirring, incorporating air into the mixture and cooling.

This is a division of application Ser. No. 10/697,800, filed Oct. 31, 2003, the contents of which are incorporated herein by reference.

### BACKGROUND OF THE INVENTION

This invention concerns gelatin-free isomaltulose-containing soft caramels and a method for producing them.

Gelatin, which is obtained on an industrial scale from collagen, primarily from bones and skin of slaughtered animals, especially cows and pigs, is one of the best-known animal products. In warm water gelatin forms a viscous solution that solidifies to a gel-like consistency below about 35° C. at a gelatin concentration of at least 1 wt %. For this reason gelatin is used in many foods as gelatinization agent, foaming agent, and binder, texturing agent and/or emulsifier. Gelatin is also characterized by easy digestibility. Gelatin determines the appearance of, for example, jellied meats and sausages, jelly foods and sweets. Gelatin seeks to improve consistency in products such as ice cream and yogurt products.

Gelatin is likewise used as a texturing agent in sweets like soft caramels. In particular the ability of gelatin to bind fat components is important here. Moreover, gelatin affects the chewability of the soft caramel mass by reducing or preventing recrystallization of caramel components, especially sugars. Gelatin also prevents agglomeration, i.e., the coalescence of small very fine crystals. Here the gelatin molecules are absorbed from the surface of the crystals and form a kind of isolating layer around the crystals, but where the nature of the crystal itself is not changed. In addition, gelatin also affects the foaming capacity of the soft caramel mass. Since gelatin is a hydrocolloid, it has a stabilizing effect due to an increase of innerlamellar binding of water.

However, gelatin-containing foods are increasingly disapproved of at least by certain groups of consumers, for various reasons. For example, many vegetarians consume only animal products such as milk, milk products and eggs, if they consume animal products at all, but they do not eat any other products that derive from animals, thus no gelatin-containing foods as well. Also, followers of kosher diets, which are relatively common in the United States, for example, and are frequently practiced even by non-Jewish consumers, reject the consumption of gelatin-containing foods. In addition, the appearance of bovine spongiform encephalopathy (BSE) disease in cows has greatly increased the demand for gelatin-free products.

However, agar, which is usually used, has the disadvantage that it must be boiled for several minutes for the products to be able to incorporate sufficient water and to be processed efficiently. In milk, products like spreads for bread, deserts, whipped products and fermented products combinations of vegetable or microbial hydrocolloids, for example, are used in order to achieve a gelatin-like activity, in particular to produce a certain texture, to achieve syneresis, thus to avoid phase separation in gels and to achieve foam stability. These combinations often consist of a mixture of gelling and nongelling substances. Starches are also frequently used as an alternative to gelatin in milk products like yogurt. Starches also form gels when heated and can store water. However, the use of starch or starch derivatives as the only gelatin substituted in milk products in some cases brings considerable problems, since for some products it is necessary to use an extraordinarily high dosage to bring about gelation. Therefore, combinations of starches and hydrocolloids are better suited for some milk products. In many milk products fiber-containing substances such as oligofructose products and wheat fiber products, mostly in combination with starches, are used to improve mouth feel and texture.

In spite of the developments cited above, the replacement of gelatin in the foods is still an extraordinarily difficult task. It turned out that with most applications a single additive does not by itself have the properties that are necessary to replace gelatin completely.

The technical problem underlying this invention is to make available gelatin-free soft caramels, where gelatin is replaced by a nonanimal substance that has properties such as low elasticity, high water dispersibility, good bodying and texturing properties, good mouthfeel and no characteristic flavor and therefore can completely replace gelatin, as well as a method for producing them.

This invention solves this technical problem by making available a gelatin-free soft caramel consisting of a soft caramel base, which contains at least one polysaccharide hydrocolloid as texturing agent, a crystalline sweetener phase formed by isomaltulose and a noncrystalline sweetener phase.

### BRIEF SUMMARY OF THE INVENTION

Surprisingly, it was established in accordance with the invention that polysaccharide hydrocolloids have properties such that enable the complete replacement of gelatin as texturing agent in soft caramels, so that the special texture and consistency of the soft caramels is retained.

Soft caramels have a soft and chewable consistency that is due to a residual water content of 6% to 10% and to characteristic recipe components of soft caramels such as fat and, up to now, gelatin. Basically, soft caramels consist of a less soluble crystalline phase, a readily soluble noncrystalline phase, and a gaseous phase enclosed in the soft caramel mass, which leads to a smooth and light nature. The noncrystalline phase in the soft caramel mass serves to inhibit the crystallization of components and to stabilize the moisture, and the noncrystalline phase also has a crucial roll in the formation of body and the strength and viscosity of the soft caramel mass and affects the chewability of the soft caramel. Soft caramels also contain a liquid phase, whose viscosity is of decisive importance for the consistency of the soft caramel. In combination with soft caramel components like fat and gelatin the phases bring about the special consistency of soft caramels, in particular a chewable short texture and prompts [sic] the consumer to chew, but not swallow, the soft caramel. In the production of traditional soft caramels the use of gelatin plays an important role, since gelatin as a texturing agent affects the viscosity of the soft caramel mass and because of this prevents the recrystallization of the soft caramel components and also has a positive effect on the stabilization of the incorporated air.

### DESCRIPTION OF THE INVENTION

In accordance with the invention it was now established that polysaccharide hydrocolloids in soft caramels, like gelatin, can bind fat, store water and stabilize the incorporated air, affect the chewability of the soft caramel in accordance with the invention by reducing or preventing recrystallization and prevent the agglomeration or coalescence of small very fine crystalline components of the soft caramel in accordance with the invention. Polysaccharide hydrocolloids also advantageously affect the foaming capacity of the soft caramel mass and in this way have a stabilizing effect. Moreover, it surprisingly turned out that the temperature stability of the isomaltulose that is used in accordance with the invention as crystalline phase is considerably improved through the replacement of gelatin by polysaccharide hydrocolloids in a soft caramel in accordance with the invention.

The gelatin-free soft caramel in accordance with the invention is also characterized in particular by the fact that the sucrose that is traditionally used in soft caramels as crystalline sweetening phase is completely replaced by isomaltulose, both from the standpoint of technology and flavor. Isomaltulose gives the gelatin-free soft caramel in accordance with the invention a sweet flavor, promotes the development of the flavor of flavorings contained in the sweet caramel and also contributes to the formation of body in the soft caramel in accordance with the invention. The isomaltulose that is used as crystalline phase in accordance with the invention is characterized by low solubility and, in connection with this, a tendency to crystallize. The strong crystallization of isomaltulose advantageously leads to an increase of the shortness of texture of the soft caramel mass. Therefore, isomaltulose, in addition to other components of the soft caramel in accordance with the invention, affects its plasticity and texture.

In connection with this invention a “soft caramel” is understood to mean a sweet that is made from a syrup, fat and, a sweetener solution by boiling. Traditional soft caramels contain approximately 30-60% sucrose, 20-50% starch syrup, 1-10% invert sugar, 0.6% lactose, 2-15% fat, 0-5% milk protein, 0-0.5% gelatin and 4-8% water. Moreover, soft caramels contain acids and flavorings. The consistency of soft caramels, which is considerably more elastic than that of hard caramels, is achieved through the higher fat and water content and through the incorporation of air. Emulsifier-containing triglycerides based on palm kernel or soy oil in particular are used as the fat component for soft caramel manufacture.

In connection with this invention “hydrocolloids” are understood to mean thickeners, swelling agents or gelling agents, which are organic high-molecular substances that can take up liquids, as a rule water, and swell. Hydrocolloids pass into viscous true or colloidal solutions and then form gels or mucilage. Thickeners have a significant effect on consistency of a food, for example by increasing the viscosity of a system, formation of a gel structure or by reducing the surface tension. Therefore, thickeners also have emulsifier activity. Thickeners can in this way stabilize solid/liquid systems like fruit nectars, liquid/liquid systems like dressings, or gas/liquid systems like whipped milk products. Moreover, thickeners also affect the positive and negative sensations that the texture of the food produces in the mouth and thus the enjoyment value of a food. Other effects of thickeners in foods are the reduction of water losses through binding of the water and thus a prolonging of the period of freshness, preventing the crystallization of food ingredients, for example sugars, and improvement of mechanical properties of foods such as firmness, elasticity and gas-holding capacity.

“Hydrocolloids based on polysaccharides” or “polysaccharide hydrocolloids” in connection with this invention are understood to mean hydrocolloids that consist of polysaccharides, in particular polysaccharides of vegetable or microbial origin. Polysaccharide hydrocolloids are therefore substances that are soluble or only dispersible in water and can swell while absorbing water, so that a viscous solution, pseudogel or gel arises. They act, for example, by stiffening the aqueous phase or by direct interactions with surface-active substances.

“Polysaccharides” are macromolecular carbohydrates whose molecules consist of a large number, in particular at least more than 10, but normally considerably more, glycosidically linked monosaccharide molecules. Polysaccharides can consist of only one type of constitutional unit, which are optionally bonded to each other in an alternating glycosidic linkage. Polysaccharides, in particular the heteroglycans that occur in vegetable gums, can also consist of different monomer units.

In a particularly preferred embodiment of the invention the polysaccharide hydrocolloid used as texturing agent in soft caramels is in particular gum arabic, gellan gum, guar gum, cellulose gum, carob seed gum, tamarind seed gum, tara gum, gum tragacanth, xanthan gum, agar, alginates, carrageen, konjac, pectin, pullulan, starches, modified starches or mixture thereof.

“Gum arabic” is the dried exudate of various species of acacia. Gum arabic is a weakly acid product that in natural form is in the form of a neutral or weakly acid K, Ca or Mg salt. The main components of gum arabic are L-arabinose, L-rhamnose, D-galactose and D-glucuronic acid. The mol ratio of these components is highly dependent on the species of acacia from which the gum arabic is obtained. Gum arabic is a branched polysaccharide whose main component consists of β-(1,3)-branched D-galactopyranose units. Gum arabic is very readily water soluble, with 1-15% solutions only having low viscosity, while higher concentrations lead to a viscous gel-like mass.

“Gellan gum” is an exocellular polysaccharide of the organism Sphingomonas elodea. The high molecular polysaccharide principally consists of a repeating tetrasaccharide unit, which consists of a rhamnose molecule, two glucuronic acid molecules and two glucose molecules, and is substituted with acyl groups, in particular glyceryl and acetyl groups. Gellan gum can form different textures that range from soft elastic gels to hard brittle gels. By mixing gellan gums with a high fraction of acyl groups and gellan gums with a low fraction of acyl groups gel structures of many different kinds can be produced. “Guar gum” is a colloidal powder that is obtained by grinding the endosperm of the seeds of the tree Cyamopsos tetragonolobus. The soluble part of guar gum is a nonionic polysaccharide of β-1,4-glycosidically linked D-mannopyranose units with α-1,6-linked D-glactopyranose in the side chain, and one D-galactose unit per 2 mannose units. As a hydrocolloid, guar gum swells in water, but without forming a clear solution. If a small amount of borax is added to guar gum solutions, gum-like gels form. Guar gum exhibits synergistic effects with other polysaccharides like agar, carrageen, starch or xanthan.

“Cellulose gums” are obtained by chemical modification of cellulose, a linear glucose-based polymer with β-1,4-linka_{g}es. Cellulose gums in clued microcrystalline cellulose (MCC), carboxymethylcellulose (CMC), methylcellulose(MC) and hydroxypropylmethylcellulose (HPMC). MCC crystals are obtained in powder or colloidal form by hydrolysis of cellulose. Although these crystals are not soluble, the colloidal form can take up water while forming thixotropic gels. The resulting gels can be used as stabilizers or fat substitutes. CMC is the sodium salt of carboxymethyl ether of cellulose with a degree of substitution from 0.4 to 0.8. The degree of substitution affects the properties of the gum, including its solubility. CMC can stabilize protein dispersions. Through the reaction of alkali cellulose with methyl chloride MC is formed, while the reaction of alkali cellulose with propylene oxide and methyl chloride leads to the formation of HPMC. The methyl celluloses that are soluble in cold water show a reversible thermal gelation, i.e., they gel under the effect of heat, while resolubilization occurs at reduced temperatures. Like CMC, the DS affects the properties of the gum, so that solid gels formed at temperatures of 50° C. become weak gels at temperatures of more than 90%.

“Carob gum” (carob seed flour) is a galactomannan from the endosperm of the seeds of the carob tree. The gum has a molecular weight from 300,000 to 360,000 and consists of a chain of β-(1,4)-linked D-mannopyranoside units, to which are bonded α-(1,6)-linked α-galactopyranoside units, where the mannose/glactose content is between 5:1 and 4:1. Presumably in the molecule there are blocks of unsubstituted mannose units, between which there are regions in which every second mannose residue has a galactose unit.

“Tamarind seed gum” (tamarind seed flour) is a hydrocolloid obtained from the seeds of the tamarind consisting of β-(1,4)-linked D-glucose units in the main chain and D-xylose, D-galactose and L-arabinose in the side chains. The molecular weight is about 50,000. Tamarind seed flour produces highly viscous solutions in cold water that gel with 65-70% sucrose even without acid. Tamarind seed flour forms stable gels over a broad pH range. These gels exhibit only slight syneresis. In contrast to pectin, these gels are also stable at lower sugar concentrations.

Tara gum is a galactomannan that occurs in the endosperm of the seed of the tara tree and has the individual constitutional units galactose and mannose in a 1:3 ratio. The molecule consists of B-(1,4)-linked D-mannopyranose units, to which D-galactopyranoside units and α-(1,6)-bonds are laterally bonded. At present the distribution of the galactose molecules in the chain is still uncertain. The physical and chemical properties to a very great extent correspond to those of the guar gum and carob seed flour. Tara gum is not completely soluble in cold water, and the solution has a considerably higher viscosity than solutions of guar gum or carob seed flour of the same concentration. Like carob seed flour, tara gum forms gels with xanthan, except that the latter are weaker and the melting point of the gels is lower. Tara gum exhibits synergistic stiffening of gel with agar and carrageen, too.

“Gum tragacanth” is an exudate from the seeds and branches of shrubs belonging to the species Astragalus. The individual components of gum tragacanth are L-rhamnose, L-fucose, D-xylose, L-arabinose, D-galactose, D-glucose and D-galacturonic acid in a ratio of 2.0:2.8:8.3:24.5:7.0:7.6:23.2. Gum tragacanth consists of 60-70% of a fraction that is swellable but not soluble in water (bassorin) and 30-40% of a water-soluble fraction, the so called tragacanthin. The water-soluble fraction is a highly branched arabinogalactan consisting of 75% L-arabinose, 10% D-galactose and 10% D-galacturonic acid. Bassorin is a highly branched molecule with a chain of α-(1,4)-linked D-galacturonic acids, which have side chains of different links in C3 positions. Tragacanth swells in water while taking up an amount of water that corresponds to 45-50 times its own weight, with the formation of tough, highly viscous mucilages, which are stable in consistency in a pH range of 2-8.

“Xanthan gum” is an exocellular heteropolysaccharide from Xanthomonas campestris with the individual components D-glucose, D-mannose and D-glucuronic acid in a ratio of 2.8:2.0:2.0. In addition, it contains about 5% acetyl and 3% pyruvyl groups. It is a β-(1,4)-glucan chain in which the position 3 of the glucose molecule is linked to a side chain that consists of two mannose units and a glucuronic acid unit. Xanthan gum is readily soluble in cold and hot water and has high pseudoplasticity. Xanthan gum can be precipitated from solution with trivalent cations. Xanthan gum is not decomposed by human digestive enzymes and is partly broken down in the large intestine by microorganisms that dwell there.

“Agar” (agar-agar) is a polysaccharide from the cell walls of numerous red algae of the species Gellidium and Gracillaria. Agar is a mixture of the gelling agarose, a linear polysaccharide with a fraction up to 70%, and the nongelling agaropectin (β-1,3-linked D-galactose units) with a fraction up to 30%. The molecular weight of agar is about 110,000-160,000. Agar is insoluble in cold water, but soluble in hot water. With a 1% solution a solid gel that melts at 80-100° C. and resolidifies at 45° C. is formed.

“Alginates” are salts of algic acid. Alginates are acidic, carboxy group-containing polysaccharides with a molecular weight of about 200,000 and consisting of D-mannuronic acid and L-glucuronic acid in different ratios, which are bonded to each other with 1,4-glycoside linkages. The Na, K, NH₄, and MG alginates are water soluble. Ca alginates form thermally irreversible gels at certain ratios. Through the acidification of aqueous alginate solutions with mineral acids the water-insoluble algic acid is precipitated. Alginates can in particular prevent the crystallizing of sugar or sugar types.

“Carrageen” [sic; carrageenan] is a group of polysaccharides that are contained in a number of types of red algae species. With regard to chemical structure, carrageen is formed similar to agar, but the fractions of the galactose sulfates are different. λ-carrageenan, κ-carrageenan and τ-carrageenan are commercially important. λ-carrageenan is a chain molecule that is formed of dimer constitutional units, namely β-1,3-D-galactose 4-sulfate and α-1,4-3,6-D-anhydrogalactose. These dimers are 1,3-glycosidically linked. The primary alcohol groups of the α-D-galactose is esterified with sulfuric acid and the hydroxy groups at C2 position of both galactose residues are likewise esterified up to about 70% with sulfuric acid.

κ- and τ-carrageenans are formed from the dimer carrabiose, in which β-D-galactose is 1,4-glycosidically linked to α-D-3,5-anhydrogalactose. These dimers are linked into a chain molecule by 1,3-glycosidic bonds. The difference between the two types of carrageenan lies in the sulfation. While in the case of κ-carrageenan the sulfate ester group is on C4 of the galactose, in the case of τ-carrageenan the hydroxy group on C2 of the anhydrogalactose is additionally esterified with sulfuric acid. The average molecular weight of carrageenan is between 100,000 and 800,000.

“Konjac” is a glucomannan that is obtained from the roots of Amorphophallus konjac. Konjac is a linear molecule formed from mannose and glucose with randomly distributed acetyl groups. In powder form it slowly swells at low temperatures. Upon treatment with alkali and heat it forms an elastic thermally irreversible gel, where the gel is stable at a pH value from 3 to 9.

“Pectins” are very common in all higher plants and are extracted in particular from the peels of citrus fruits and apple peels. The primary individual components of pectins are D-galacturonic acid. In addition, they contain as secondary components L-rhamnose, D-galactose, L-arabinose and D-xylose. The pectin molecule consists of a chain of (1,4)-linked α-D-galacturonic acid units that are interrupted by L-rhamnose units whose 1-2 positions are bonded to each other. In addition, D-galactose, D-xylose and L-arabinose units can occur as side chains. The molecular weight of extracted pectins is an average of 100,000 and is highly dependent on the extraction conditions that are used in each case. High- and low-esterified pectins as well as the alkali salts of pectic acid are soluble in water, while pectic acid is insoluble in water. Through the formation of hydrogen bridges associations occur in partial regions of the pectin chain, so that three-dimensional network form.

“Pullulan” is an exocellular polysaccharide of the yeast-like fungus Aureobasidium pullulans. Pullulan is a homopolysaccharide with D-glucose as the only constitution unit. In chains multotriose units are bonded to each other by α-1,6-linkages. The molecular weight of pullulan is 10,000-400,000.

In a particularly preferred embodiment of the invention a mixture of gum arabic and gellan gum is used as polysaccharide hydrocolloid. Preferably the ratio between arabic and gellan gum is 5:1 to 15:1.

It is foreseen in accordance with the invention that the amount of polysaccharide hydrocolloid or mixture thereof in the total amount of the soft caramel base mass is about 0.4% to about 0.8%, preferably about 0.6%, with respect to the dry weight of the soft caramel base mass.

It is further provided in accordance with the invention that the fraction of the isomaltulose that forms the crystalline phase in the total amount of the soft caramel base mass is about 35% to about 70%, preferably about 42% up to about 65%, with respect to the dry weight of the soft caramel base mass.

In another preferred embodiment of the invention the gelatin-free soft caramel is a sugar-free gelatin-free soft caramel, where the noncrystalline sweetener phase of the soft caramel base mass is formed of maltitol syrup, polydextrose and/or hydrogenated starch hydrolysate. In another preferred embodiment of the invention the gelatin-free soft caramel in accordance with the invention is a sugar-containing gelatin-free soft caramel, where the noncrystalline sweetener phase of the soft caramel base mass is formed of a glucose syrup or starch hydrolysate.

It is likewise provided in accordance with the invention that the gelatin-free hard [sic] caramel in accordance with the invention can contain, besides the said types of sugar and/or sugar substitutes, additionally one or more intensive sweeteners. Intensive sweeteners are compounds that are characterized by an intensive sweet flavor while having low or negligibly low food value. In accordance with the invention it is especially provided that the intensive sweetener is cyclamate, for example sodium cyclamate, saccharine, aspartame, glycyrrhizin, neohesperidine dihydrochalcone, thaumatin, monellin, acesulfame, alitame or sucralose.

It is further provided in accordance with the invention that the soft caramel base mass of the gelatin-free soft caramel contain 2-15% fat. Preferably the fat contained in the gelatin-free soft caramel in accordance with the invention is hydrogenated palm kernel fat.

In another embodiment of the invention it is provided that the soft caramel base mass of the gelatin-free soft caramel contain at least one emulsifier. An “emulsification agent” or “emulsifier” is understood to mean an auxiliary substance that is used in manufacture and for stabilization of emulsions. Emulsifiers are surface-active substances that reduce the interfacial tension between the two phases oil and water and, besides reducing the interfacial energy, also produce a stabilization with the emulsion that is formed. Emulsifiers stabilize the emulsion through interfacial films and through the formation of steric or electrical barriers, due to which the merging of the emulsified particles is prevented. Both the elasticity and viscosity of the interfacial films are important factors in emulsion stabilization and are highly affected by the emulsifier.

In another embodiment of the invention it is provided that the soft caramel base mass of the gelatin-free soft caramel contains 0% to 5% of at least one protein component. The protein component in accordance with the invention can be a protein of animal, vegetable or microbial origin. Preferably, the protein component is in particular milk protein.

In still another embodiment of the invention it is provided that the soft caramel base mass of the gelatin-free soft caramel in accordance with the invention contain one or more natural or synthetic food dyes. In connection with this invention a “food dye” is understood to mean a substance that is used in the manufacture of foods for purposes of color correction or to produce a pleasant appearance. Food dyes make a considerable contribution to the acceptance of foods. The food dyes used in accordance with the invention can be both of natural and of synthetic origin. Among natural food dyes are dyes of vegetable origin, for example carotenoids, flavonoids and anthocyans, dyes of animal origin, for example, cochineal, and inorganic pigments like titanium dioxide, iron oxide pigments and iron hydroxide pigments. Food dyes also include products of enzymatic browning like polyphenols and products of nonenzymatic browning like melanoidines as well as products of heating, for example sugar coloring and caramel. Synthetic food dyes include in particular azo, triphenylmethane, indigoid, xanthene and quinoline compounds.

In a preferred embodiment of the invention the dyes are chlorophyllin, carmine, red, alura red, β-carotene, riboflavins, anthocyans, betanine, erythrosine, indigo carmine, tartrazine or titanium dioxide.

Of course, the soft caramel base mass of the gelatin-free soft caramel in accordance with the invention can contain additional flavorings and flavorings agents. Such substances are, for example, essential oils, synthetic flavorings or mixtures thereof, for example oils from plants or fruits like citrus oil, fruit essences, peppermint oil, clove oil, anise, crystalline acid, menthol, eucalyptus, etc.

It is provided in accordance with the invention that the water content of the soft caramel mass of the gelatin free soft caramel in accordance with the invention amounts to 5 to 14% water, especially 6 to 12% water, preferably 6 to 8%.

In another embodiment it is provided that the soft caramel base mass of the gelatin-free soft caramel in accordance with the invention additionally contains a medicinal active agent, for example dextromethorphan, hexylresorcinol/menthol, phenylpropanolamine, dyclonine, menthol eucalyptus, benzocaine or cetylpyridinium.

The gelatin-free soft caramels in accordance with the invention can be in the form of both filled and unfilled caramels, where the soft caramels in accordance with the invention can contain all of the fillings known in the prior art. Of course, the gelatin-free soft caramels in accordance with the invention can also be in coated or uncoated form, where the coating thickness that are usually used in the prior art to produce coated soft caramels can be used.

This invention also solves the technical problem that underlies it by a method for producing a gelatin-free, isomaltulose-containing soft caramel that consists of

a) preparation of a noncrystalline sweetener phase by dissolving at least one soluble sweetener in water,

b) addition of at least one polysaccharide hydrocolloid, at least one fat component, at least one emulsifier and a part of the total amount of the isomaltulose that forms the crystalline sweetener phase to the noncrystalline sweetener phase,

c) heating the mixture obtained in (b) to a temperature of at least 100° C. by feed of steam,

d) addition of the remaining isomaltulose to the heated mixture while stirring,

e) incorporation of air into the mixture obtained in (d) and

f) cooling the mixture.

In a preferred embodiment of the invention it is provided that about 70% to 90% of the total amount of isomaltulose is added to the prepared noncrystalline sweetener phase and then they are heated together. Preferably about 74% to 85% of the total amount of isomaltulose is added to the noncrystalline sweetener phase and they are then heated together.

In a preferred embodiment the mixture formed by mixing the noncrystalline sweetener phase and the fat component, the polysaccharide hydrocolloid, the emulsifier and a part of the total amount of the isomaltulose is heated to a temperature of 110° C. In a preferred embodiment the feed of steam is stopped after heating the mixture containing the noncrystalline sweetener phase and the mixture is subjected to a vacuum. After the end of the [sic; treatment] steam temperature the temperature of the mixture then rises to 125° C. to 130° C. Then the batch cooker that is preferably used to cook the mixture is opened and the remaining isomaltulose is added to the heated mixture while stirring. The introduction of air into the resulting mixture can take place by beating the air into the heated mixture after adding the remaining isomaltulose. In an alternative embodiment the mixture obtained after adding the remaining isomaltulose is first cooled and then the air is introduced into the mixture by pulling the cooled mixture. Then a strand is drawn from the whipped cooled mass or the pulled cooled mass and from it the corresponding soft caramel pieces are cut into the desired size. Preferably the cut pieces have a weight of 2 to 7 g. The resulting soft caramels can then be packaged using the conventional methods for soft caramels, for example wrapping or enveloping.

The invention is illustrated in more detail by the following examples.

### EXAMPLE 1

### Preparation of the Gelation-Free Isomaltulose-Containing Soft Caramels

| | g in batch | in % |
|---|---|---|
| Raw material I | | |
| Water | 267.00 | 7.93 |
| Polydextrose solution with | 1753.54 | 52.06 |
| 75% solids | | |
| Hydrogenated palm kernel | 192.00 | 5.70 |
| fat | | |
| Gum arabic | 14.44 | 0.43 |
| Gellan gum | 1.60 | 0.05 |
| Isomaltulose | 832.89 | 24.73 |
| Emulgator E 471 | 19.50 | 0.58 |
| Aspartame/Acesulfame K | 0.32 | 0.01 |
| Raw material II | | |
| Isomaltulose, finely | 287.24 | 8.53 |
| ground, type PF | | |
| Total | 3368.53 | 100.00 |

The polydextrose powder and water are mixed with a whisk. Then all the other components of raw material class I are put into a batch cooker and stirred with a stirrer for 3 min. Then the mass is heated. At 110° C. the feed of steam is stopped and a vacuum is applied for about 2 min. After the steam has been stopped, the mass heat up further to 125° C. to 130° C. Then the batch cooker is opened. The components of raw material class II are added and stirred for 3 min with a stirrer. For cooling the mass is transferred to a cooling table. After cooling the mass is pulled with a pulling machine for about 3 min in order to incorporate air. Then a strand is drawn from the pulled mass and pieces about 2 to 7 g are cut from it. The resulting soft caramels can be packaged by the methods that are usable for soft caramels, for example wrapping or enveloping. The water content of the soft caramels obtained by this method is 6 to 12 g/100 g of total amount.

### EXAMPLE 2

### Preparation of Gelatin-Free Soft Caramels

| | g in batch | in % |
|---|---|---|
| Raw material I | | |
| Water | 267.00 | 7.92 |
| Polydextrose solution with | 1034.57 | 30.70 |
| 75% solids | | |
| Hydrogenated palm kernel | 192.00 | 5.70 |
| fat | | |
| Gum arabic | 16.04 | 0.48 |
| Gellan gum | 1.60 | 0.05 |
| Isomaltulose | 1551.86 | 46.05 |
| Emulgator E 471 | 19.50 | 0.58 |
| Aspartame/Acesulfame K | 0.32 | 0.01 |
| Raw material II | | |
| Isomaltulose, finely | 287.24 | 8.52 |
| ground, type PF | | |
| Total | 3370.13 | 100.00 |

The gelatin-free soft caramels were prepared by analogy with Example 1.

1. A method for producing a gelatin free isomaltulose containing soft caramel that comprises
a) preparing a noncrystalline sweetener phase by dissolving at least one soluble sweetener in water,
b) adding at least one polysaccharide hydrocolloid, at least one fat component, at least one emulsifier, and a part of the total amount of a crystalline sweetener phase to the noncrystalline sweetener phase, wherein the crystalline sweetener phase is formed by isomaltulose,
c) steam heating the mixture obtained in (b) to a temperature of at least 100° C.,
d) adding the remaining isomaltulose to the heated mixture while stirring,
e) incorporating air into the mixture obtained in (d) and
f) cooling the mixture.

2. A method as in claim 1, where 70% to 90% of the total amount of isomaltulose is added to the noncrystalline sweetener phase.

3. A method as in claim 2, where 74% to 85% of the total amount of isomaltulose is added to the noncrystalline sweetener phase.

4. A method as in claim 3, where the mixture containing the noncrystalline sweetener phase is heated to 110° C.

5. A method as in claim 1, where the mixture containing the noncrystalline sweetener phase is heated to 110° C.

6. A method as in claim 1, where after heating the mixture containing the noncrystalline sweetener phase, the steam heating is stopped and the mixture is subjected to a vacuum.

7. A method as in claim 6, where after the end of the steam heating, the temperature of the mixture rises to 125° C. to 130° C.

8. A method as in claim 1, where after adding the remaining isomaltulose, the air is introduced into the mixture by whipping the heated mixture.

9. A method as in claim 1, where after adding the remaining isomaltulose, the heated mixture is cooled and air is introduced into the mixture by pulling the cooled mixture.

10. A method as in claim 1, where the air containing mixture after cooling is pulled to a strand and the strand is cut into pieces.

11. A method as in claim 1, where the polysaccharide hydrochloride is selected from the group consisting of gum arabic, gellan gum, guar gum, cellulose gum, carob seed gum, tamarind seed gum, tara gum, gum tragacanth, xanthan gum, agar, alginate, carrageenan, konjac, pectin, pullulan, a starch, a modified starch and a mixture thereof.

12. A method as in claim 1, where the polysaccharide is a mixture of gum arabic and gellan gum in a ratio from 5:1 to 15:1.

13. A method as in claim 1, where the noncrystalline sweetener is maltitol syrup, polydextrose or hydrogenated starch hydrolysate or a mixture of two or three thereof, and no sucrose is used in the method.

14. A method as in claim 1, where the noncrystalline sweetener is glucose syrup or starch hydrolyzate or both, and sucrose is used in the method.

15. A method as in claim 1, in which at least one intensive sweeteners selected from the group consisting of cyclamate, saccharine, aspartame, glycyrrhizin, neohesperidine dihydrochalcone, thaumatin, monellin, acesulfame, alitame and sucralose is incorporated into the mixture.

16. A method as in claim 1, where the amount of the fat is 2 to 15% fat of the soft caramel product.

17. A method as in claim 1, in which b) includes adding a positive amount up to 5% of at least one protein component, and at least one natural or synthetic food dyes.

18. A method as in claim 17, wherein the food dye is selected from the group consisting of chlorophyll, a chlorophyllin, carmine red, alura red, β carotene, a riboflavin, an anthocyan, betanine, erythrosine, indigo carmine, tartrazine or titanium dioxide.

19. A method as in claim 1, in which b) includes adding flavorings and flavoring agents.

20. A method as in claim 1, in which at least one medicinal active agent selected from the group consisting of dextromethorphan, hexylresorcinol/menthol, phenylpropanolamine, dyclonine, menthol eucalyptus, benzocaine or cetylpyridinium is incorporated into the mixture.
